(19) United States
(12) Patent Application Publication
(10) Pub. No.: US 2006/0266366 A1
(43) Pub. Date: Nov. 30, 2006

(54) Illumination means for catheter placement

(76) Inventors: Ross Tsukashima, San Diego, CA (US); Erich H. Wolf, Vista, CA (US); Jeffery D. Schipper, Ramona, CA (US); Charles S. Bankert, Oceanside, CA (US); Leo R. Roucher, Rancho Santa Fe, CA (US)

Correspondence Address: MICHAEL E. KLICPERA, PO BOX 573, LA JOLLA, CA, 92038-0573, US

(21) Appl. No.: 11/137,619

(22) Filed: May 25, 2005

(51) Int. Cl. A62B 9/06; A61M 16/00

(52) U.S. Cl. 128/207.14; 128/207.15; 128/207.16; 128/200.26

(57) ABSTRACT

The present invention is a system comprising an illumination means and is mounted on a catheter wherein said illumination is generally located in the distal end of the catheter. Locating the illumination means near the distal end will simplify and facilitate precise placement of a measurement device in close proximity to the desired location. In one example, a catheter with a distally mounted pH sensor benefits from the use of an illumination means in the patient's airway such as in the oropharynx region. The illumination source of the present invention addresses catheter insertion and location using a continuous or flashing light emitting diode (LED) embedded in the distal end of the catheter to provide a visual sighting means for the physician. When the catheter, with the illumination means is inserted in the patient's airway, the illumination means functions to illuminate the distal end of the catheter and the anatomical features of the patient's airway for observation by the clinician, thereby facilitating proper placement of the measurement device.

### FIELD OF THE INVENTION

The field of art to which this invention relates to is placement of various catheter devices within certain corporal pathways. More specifically, this invention relates an apparatus and method of accurately positioning a catheter at a specified location within a patient's airway.

### BACKGROUND OF THE INVENTION

Very often clinicians, when providing treatment to patients, must insert and navigate various types of catheters into a variety of pathways in order to diagnose or monitor parameters known to be associated with certain disease conditions or to provide treatment for such disease conditions. In many cases, visualization of the catheter's placement within the body is essential to assessing a patient's condition or administering therapy. Typical placement methods use radiography techniques when the catheter cannot be observed with the naked eye and if the particular catheter device has sufficient density to be radio-opaque. Even if the catheter device has the required density and properties to be radio-opaque, cumbersome equipment emitting unhealthful X-rays or other potentially detrimental radiofrequencies are required. Ultrasound techniques are another means to locate various organs, tissues and devices but complex and expensive additional equipment is required. Furthermore, ultrasonic digital pictures generally do not have absolute clarity and artifacts can be generated. A simple method commonly used is to provide a series of marking bands or indicators along the catheter's shaft. However, this method does not compensate for the anatomical difference between patients.

Accordingly, there is a need for a novel illumination means associated with a catheter apparatus for accurately placing the catheter within a specified location that compensates for anatomical differences, is cost effective, and does not require the use of additional complex equipment.

### SUMMARY OF THE INVENTION

The present invention is a system comprising an illumination means that is mounted on a catheter wherein said illumination is generally located in the distal end of the catheter. Locating the illumination means near the distal end simplifies and facilitates precise placement of a measurement device in close proximity to the desired location. In one example, a catheter with a distally mounted pH sensor benefits from the use of an illumination means in the patient's airway such as in the oropharynx region. The illumination source of the present invention addresses catheter insertion and location using a continuous or flashing light emitting diode (LED) embedded in the distal end of the catheter to provide a visual sighting means for the physician. When the catheter with the illumination means is inserted in the patient's airway, the illumination means functions to illuminate the distal end of the catheter and the anatomical features of the patient's airway for observation by the clinician, thereby facilitating proper placement of the measurement device.

It is an objective of the present invention to provide a catheter apparatus and method for accurately placing the catheter within a specified location in a patient's airway that compensates for anatomical differences.

It is also an objective of the present invention to provide a catheter apparatus and method that can more accurately place a catheter within a patient's airway without the need of additional complex equipment.

These and other features, aspects and advantages of the present invention will become better understood with reference to the following descriptions and claims.

### BRIEF DESCRIPTION OF THE DRAWINGS

FIG. 1 is a perspective representation of the present invention showing the entire catheter length with an illumination means and a measurement means located on the distal end of the catheter and a power source located on the proximal end of the catheter.

FIG. 2 is an example of the first embodiment of the present invention comprising a sectional view of a tear-drop shaped structure mounted on the exterior surface of the distal end of the catheter, wherein an illumination means is mounted in the tip of the catheter.

FIG. 3 is another embodiment of the present invention showing the distal end of a typical catheter device whereby an illumination means is embedded within the tip of the catheter.

### DESCRIPTION OF THE PREFERRED EMBODIEMENTS

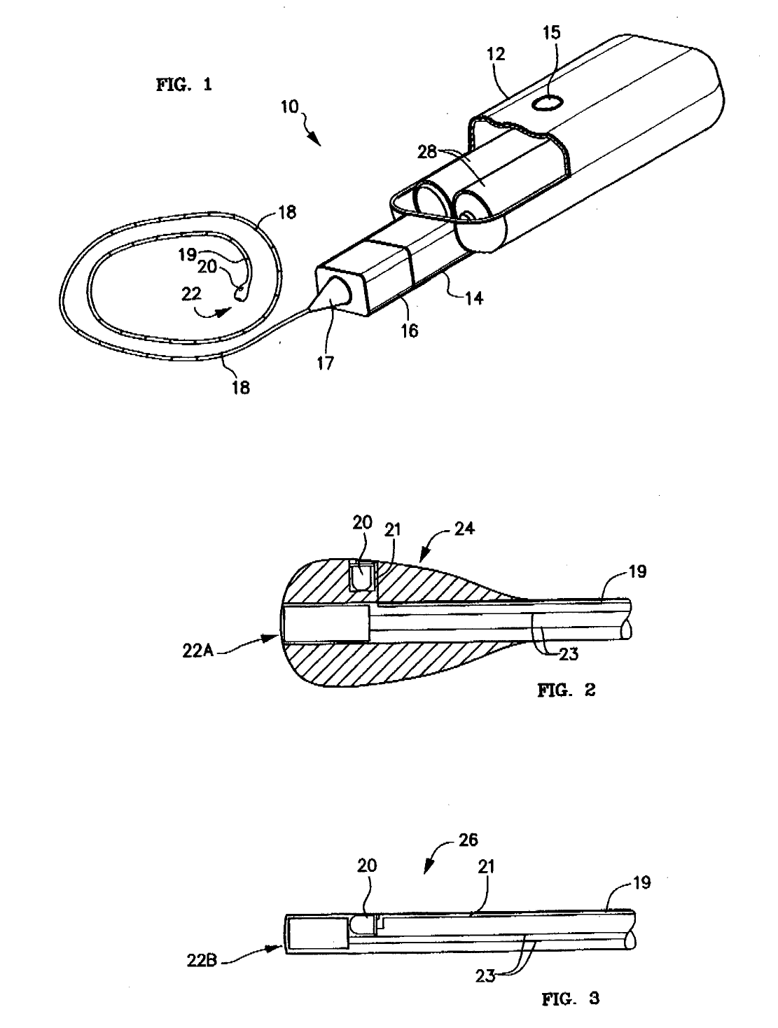

The present invention is a system comprising an illumination means typically mounted in close proximity to the distal end of a general catheter system. Optionally, the catheter system can have mounted near or within the distal end of the catheter, a measurement device 22a, 22b, such as a pH sensor. FIG. 1 shows the present invention system 10 with the entire length of the catheter 18 and the illumination means 20 mounted in close proximity to the distal end 19 of the catheter 18. The optional measurement device 22a, 22b such as a pH sensor can also be located on the distal end 19 of the catheter 18. The catheter 18 can be a single or a multi-luminal design for allowing electrical connection from the illumination means, and optionally from the measurement device, to extend throughout the longitudinal length of the catheter and terminate in the handle 12. The outer tubular member of the catheter 18 generally has an outside diameter in the range of 0.030″ to 0.090″, and preferably between 0.050″ and 0.070″. Its wall thickness is typical for its diameter and generally is in the range of 0.005″ to 0.020″ and preferably between 0.010″ and 0.015″. The materials used to fabricate the catheter are typical thermoplastic polymers such as polyethylene, polyether block amide, polypropylene, polyvinyl chloride (PVC), polystyrene, ABS, nylon, delrin, polyethylene terephthalate (PET), polytetrafluoroethylene (PTFE), polyurethane composites, and elastomeric materials.

A handle 12 which contains a power source 28 is located on the proximal end 17 of the catheter 18. The handle 12 shows a cut away where the power source 28 for the illumination means 20 is located. As represented in this figure, a plurality of batteries is being used as the power source 28. However, the power source 28 could be hard wired to a wall socket having approximately 120 volts which is coupled to appropriate circuitry, such as step down transformers, and diode protection circuits, that is incorporated within the handle 12. The handle 12 is fitted with a switch 15, which is represented in FIG. 1 as a typical push-button type, but it is anticipated by the Applicants that other switch types, e.g. toggle, could be used. In addition, in the embodiment where an optional measurement device 22a, 22b is being used, specific circuitry (not shown) can be incorporated within the handle 12, for example, to receive and transmit measurement data collected from the measurement device 22a, 22b. Such wireless communication can be integrated so that an external command can be used to control the illumination. The handle 12 can be manufactured in various sizes and shapes in accordance with the design requirements, e.g. size of batteries, and can be fabricated by a machining or molding means using a variety of polymeric materials including polyethylene, polypropylene, polyvinyl chloride, epoxy, polyurethane, polycarbonate, acrylic, polystyrene, ABS, nylon, delrin, polyethylene terephthalate (PET), polyether block amide, fluorinated ethylene-propylene (FEP) or polytetrafluoro-ethylene (PTFE).

A connector 16 on the catheter is shown engaged to a receiving connector 14 on the handle 12. The catheter connector 16 is shown having a release mechanism 17. The catheter connector 16 and handle connector 14 utilize standard connector technology which is not of particular importance to the present invention.

The preferred illumination source 20 utilizes light emitting diode (LED) technology which has the advantages of low power requirements with sufficient candle power, relatively small physical size, and long life. However, the Applicant's anticipate that other illumination technologies might also be utilized in the present invention. Such light sources include, but are not limited to, incandescence, fluorescence, halogen and halide technologies. The illumination source 20 could be used in conjunction with the external markings along the length of the catheter. An example of this is that the clinician could first visually place the distal end 19 of the catheter 18 using the illumination means 20 and then observe the distance the catheter was placed, measured at the end of the nares (nose) with the catheter markings. This would allow clinicians to quickly verify that the catheter apparatus has remained in place during the course of a study.

FIG. 2 is an example of the first embodiment of the present invention comprising a sectional view of a tear-drop shaped structure mounted on the exterior surface of the distal end 19 of the catheter 18, wherein an illumination means 20 is mounted within the tear-drop structure 24. In this embodiment, the optional measurement device 22 is mounted flush with said distal end 19 of the catheter 18. The illumination means 20 is adhered to the tear-drop shaped structure 24 using general adhesive technology. The tear-drop shaped structure 24 is adhered to the outside surface of the catheter 18 using general adhesive technology. The distal end of the tear-drop shaped structure 43 generally has an outside diameter in the range of 0.040″ to 0.250″, and preferably between 0.100″ and 0.150″. The outside diameter then slopes towards the proximal end of the tear-drop shaped structure 24 where it approximates the outside diameter of the catheter 18. The tear-drop shaped structure 24 is usually fabricated by machining or molding means using a variety of polymeric materials including polyimide, polyethylene, polypropylene, polyvinyl chloride, epoxy, polyurethane, polycarbonate, acrylic, polystyrene, ABS, nylon, delrin, polyethylene terephthalate (PET), polyether block amide, fluorinated ethylene-propylene (FEP) or polytetrafluoro-ethylene (PTFE). As shown in this figure, to simplify placement within an airway of a patient, an embedded illumination source 20 is located in close proximity to the distal end 19 of the catheter 18. The illumination source 20 functions to illuminate the distal end of the catheter and the anatomical features of the patient's airway for observation by the clinician, thereby facilitating proper placement of the measurement device. The illumination source 20 is connected to an electrical wiring means 21 that extends the length of the catheter, is incorporated as an element of the connectors 14 and 16, and is connected to a power source in the handle 12. As discussed, the embedded illumination source 20 preferably is comprised of a light emitting diode (LED), which can be illuminated continuously or in a flashing mode to aid in determining the location of the distal end 19 of catheter 18. The optional measurement device 22 is connected to an electrical wiring means 23 that extends the length of the catheter, is incorporated as an element of the connectors 14 and 16, and is connected to optional circuitry (not shown) in the handle 12. The wiring means comprises standard wire technology where generally a conductive metallic core, e.g. copper is surrounded with a non-conductive sheath, e.g. a polymer.

FIG. 3 is another embodiment of the present invention comprising a sectional view of the distal end 19 of the catheter 18, wherein an illumination means 20 is mounted within the catheter shaft. In this embodiment, the optional measurement device 22 is mounted such that the tip of the measurement device 22 is recessed. As shown in this figure, the illumination means 20 is mounted in the catheter shaft just proximal to the optional measurement device 22 and is adhered to the shaft using general adhesive technology. Alternately, the illumination means 20 can be mounted in the catheter shaft distal to (in front of) the optional measurement device 22 and is adhered to the shaft using general adhesive technology (not shown). In addition, the illumination means 20 can be mounted in various positions in close proximity to the distal end 19 of the catheter 18 when there is no optional measurement device 20 used or mounted with the present invention. The illumination means 20 is mounted within the catheter shaft using general adhesive technology. As shown in this figure, to simplify placement within an airway of a patient, an embedded illumination source 20 is located within the distal tubular member of the catheter 18. The illumination source 20 functions to illuminate the distal end of the catheter and the anatomical features of the patient's airway for observation by the clinician, thereby facilitating proper placement of the measurement device. The illumination source 20 is connected to an electrical wiring means 21 that extends the length of the catheter, incorporated as an element of the connectors 14 and 16, and is connected to a power source in the handle 12. As discussed, the embedded illumination source 20 preferably is comprised of a light emitting diode (LED), which can be illuminated continuously or in a flashing mode to aid in determining the location of the distal end 19 of catheter 18. The optional measurement device 22 is connected to an electrical wiring means 23 that extends the length of the catheter, incorporated as an element of the connectors 14 and 16, and is connected to optional circuitry (not shown) in the handle 12. The wiring means comprises standard wire technology where generally a conductive metallic core, e.g. copper is surrounded with a non-conductive sheath, e.g. a polymer.

We Claim:

1. An illuminating device attached to a catheter comprising an apparatus, said apparatus comprising:
a catheter having a distal end, a proximal end, and at least one lumen that extends along the longitudinal length of said catheter and communicating with said distal end and said proximal end, a portion of said catheter designed to enter a nasal cavity of a patient;
an illumination means located in close proximity to said distal end, said illumination means has the function of illuminating the distal end of said catheter apparatus and the anatomical features of a patient's airway;
a power source attached to a proximal end of said catheter;
said illumination means in electrical communication with said power source;

2. The illuminating apparatus as recited in claim 1, further comprising a measurement device located in close proximity to said distal end of said catheter.

3. The illuminating apparatus as recited in claim 2, wherein said measurement device is a pH sensor.

4. The illuminating apparatus as recited in claim 2, wherein said measurement device is in electrical communication with a connector on said proximal end of said catheter.

5. The illuminating apparatus recited in claim 1, wherein said illumination means comprises light emitting diode (LED) technology.

6. The illuminating apparatus recited in claim 1, wherein said illumination means comprises incandescence light technology.

7. The illuminating apparatus recited in claim 1, wherein said illumination means comprises fluorescence light technology.

8. The illuminating apparatus recited in claim 1, wherein said illumination means comprises halogen light technology.

9. The illuminating apparatus recited in claim 1, wherein said illumination means comprises halide light technology.

10. An illuminating device attached to a sensing catheter comprising a monitoring apparatus, said apparatus comprising:
a catheter having a distal end, a proximal end, and at least one lumen that extends along the longitudinal length of said catheter and communicating with said distal end and said proximal end, a portion of said catheter designed to enter a nasal cavity of a patient;
a measurement device positioned along said longitudinal length of said catheter
an illumination means located in close proximity to said measurement device, said illumination means has the function of illuminating said measurement device and the anatomical features of a patient's airway;
a power source attached to a proximal end of said catheter; and
said illumination means in electrical communication with said power source.

11. The illuminating apparatus as recited in claim 10, wherein said measurement device is a pH sensor.

12. The illuminating apparatus as recited in claim 10, wherein said measurement device is in electrical communication with a connector on said proximal end of said catheter.

13. The illuminating apparatus recited in claim 10, wherein said illumination means comprises light emitting diode (LED) technology.

14. The illuminating apparatus recited in claim 10, wherein said illumination means comprises incandescence light technology.

15. The illuminating apparatus recited in claim 10, wherein said illumination means comprises fluorescence light technology.

16. The illuminating apparatus recited in claim 10, wherein said illumination means comprises halogen light technology.

17. The illuminating apparatus recited in claim 10, wherein said illumination means comprises halide light technology.
